(19) United States
(12) Patent Application Publication
(10) Pub. No.: US 2021/0276012 A1
(43) Pub. Date: Sep. 9, 2021

(54) DEVICE AND METHOD FOR DETECTING NUCLEIC ACIDS BY ISOTHERMAL AMPLIFICATION TECHNIQUE

(71) Applicant: TDK Corporation, Tokyo (JP)

(72) Inventors: Fei ZHOU, Dongguan (CN); Xianwen FENG, Dongguan (CN); Zhongjun WAN, Dongguan (CN); Yu FU, Dongguan (CN)

(73) Assignee: TDK Corporation, Tokyo (JP)

(21) Appl. No.: 17/257,421

(22) PCT Filed: Jul. 9, 2019
(86) PCT No.: PCT/IB2019/055818

§ 371 Date: Dec. 31, 2020

(30) Foreign Application Priority Data
Jul. 10, 2018 (CN) 201810751823.6

(51) Int. Cl. B01L 3/00; C12Q 1/6825; C12Q 1/686

(52) U.S. Cl. CPC B01L 3/502715; C12Q 1/686; C12Q 1/6825

(57) ABSTRACT

A device and a method for detecting nucleic acids by an isothermal amplification technique including a sample processor and a magneto-sensitive detector, wherein the sample processor includes a microfluidic tank, a temperature controller, a capture chip storage chamber, a DNA-modified magnetic bead storage chamber and a cleaning solution storage chamber. The detecting nucleic acids using the device have a fast reaction speed and stable signal output.

### TECHNICAL FIELD

The present invention relates to a device and a method for detecting nucleic acids, and in particular to a device and a method for detecting nucleic acids by an isothermal amplification technique.

### BACKGROUND ART

Ordinary PCR (polymerase chain reaction) amplification and fluorescence detection are the existing conventional methods for detecting DNA. A conventional PCR instrument currently used has up to dozens of PCR reaction tanks that can be precisely controlled, which allows the PCR instrument to simultaneously carry out

PCR reactions in large numbers under different reaction conditions. However, it is time-consuming and expensive for fast and simple DNA detection. Therefore, the isothermal amplification technique is being used more and more. The isothermal amplification technique is used for carrying out nucleic acid amplification under an isothermal condition in a short time and, compared with the conventional PCR technique, does not require complex processes such as thermal denaturation of DNA templates and thermal cycling, and thus is simple and fast, etc.

However, the fluorescence detection technique is still the main detection technique with respect to both the existing PCR and isothermal amplification techniques, but fluorescence signals are unstable and easy to attenuate, and the reagent storage conditions are relatively rigorous.

### SUMMARY OF THE INVENTION

An object of the present invention is to provide a device and a method for detecting nucleic acids with a fast reaction speed and stable signal output in order to overcome the deficiencies of the prior art mentioned above.

In order to achieve the above object, the present invention adapts the technical solution as follows: a device for detecting nucleic acids, wherein the device comprises a sample processor and a magneto-sensitive detector, the sample processor comprises a microfluidic tank, a temperature controller, a capture chip storage chamber, a DNA-modified magnetic bead storage chamber and a cleaning solution storage chamber;
the microfluidic tank is provided with a reagent inlet and a reagent outlet; the temperature controller is disposed on the microfluidic tank;
an inlet of the capture chip storage chamber is in communication with the reagent outlet of the microfluidic tank through a first micro-channel, is in communication with the DNA-modified magnetic bead storage chamber through a second micro-channel and is in communication with the cleaning solution storage chamber through a third micro-channel; the first micro-channel, the second micro-channel and the third micro-channel are provided with valves to control a reagent in the microfluidic tank, a DNA-modified magnetic bead and a cleaning solution to flow into the capture chip storage chamber, respectively; and
the magneto-sensitive detector comprises a magnetic sensor and a groove for accommodating the capture chip storage chamber, the capture chip storage chamber is inserted inside the groove, and the magnetic sensor senses the DNA-modified magnetic bead in the capture chip storage chamber and converts a magnetic signal of the DNA-modified magnetic bead into an electrical signal.

The present invention adapts another technical solution as follows: a device for detecting nucleic acids, wherein the device comprises a sample processor and a magneto-sensitive detector, the sample processor comprises a microfluidic tank, a temperature controller, a capture chip storage chamber, a DNA-modified magnetic bead storage chamber and a cleaning solution storage chamber;
the microfluidic tank is provided with a reagent inlet and a reagent outlet; the temperature controller is disposed on the microfluidic tank;
an inlet of the capture chip storage chamber is in communication with the reagent outlet of the microfluidic tank through a first micro-channel, is in communication with the DNA-modified magnetic bead storage chamber through a second micro-channel and is in communication with the cleaning solution storage chamber through a third micro-channel; the first micro-channel, the second micro-channel and the third micro-channel are provided with valves to control a reagent in the microfluidic tank, a DNA-modified magnetic bead and a cleaning solution to flow into the capture chip storage chamber, respectively; and
the magneto-sensitive detector comprises a magnetic sensor, and the magnetic sensor senses the DNA-modified magnetic bead in the capture chip storage chamber and converts a magnetic signal of the DNA-modified magnetic bead into an electrical signal.

Preferably, wherein the magneto-sensitive detector is located within a groove for accommodating the capture chip storage chamber.

Further, the sample processor further comprises a receiving chamber for DNA to be tested and a nuclease receiving chamber, and both of the receiving chamber for DNA to be tested and the nuclease receiving chamber is provided with a reagent inlet and a reagent outlet; the reagent inlet of the microfluidic tank is in communication with the reagent outlet of the receiving chamber for DNA to be tested through a fourth micro-channel and is in communication with the reagent outlet of the nuclease receiving chamber through a fifth micro-channel; and the fourth micro-channel and the fifth micro-channel are provided with valves to respectively control a DNA to be tested and a nuclease to flow to the microfluidic tank, respectively.

Further, both of the receiving chamber for DNA to be tested and the nuclease receiving chamber are disposed above the microfluidic tank; and the capture chip storage chamber is located below the DNA-modified magnetic bead storage chamber and the cleaning solution storage chamber, and the height of the capture chip storage chamber is not higher than that of the microfluidic tank.

Further, the sample processor further comprises a pressurizer which is respectively connected to the receiving chamber for DNA to be tested, the nuclease receiving chamber, the DNA-modified magnetic bead storage chamber and the cleaning solution storage chamber.

Further, the device for detecting nucleic acids further comprises a DNA extraction chamber having DNA extraction solution therein, and the DNA extraction chamber is in communication with the reagent inlet of the receiving chamber for DNA to be tested.

Further, the sample processor further comprises an RNA extraction chamber having an RNA extraction solution therein and a reverse transcription reagent storage chamber, and the reverse transcription reagent storage chamber is respectively in communication with a reagent outlet of the RNA extraction chamber and the reagent inlet of the receiving chamber for DNA to be tested.

Further, the temperature controller comprises a heating body, a temperature sensor electrically connected to the heating body and detecting the temperature of the heating body, and a temperature control unit electrically connected to the heating body and controlling the temperature of the heating body; the temperature sensor is further electrically connected to the temperature control unit for transferring the detected temperature of the heating body to the temperature control unit; and the heating body is disposed on the microfluidic tank.

Further, the sample processor further comprises a waste solution cell that is in communication with an outlet of the capture chip storage chamber, and a pipe, connecting the waste solution cell with the outlet of the capture chip storage chamber is provided with a valve.

Further, the valve is a mechanical valve or a solenoid valve.

Further, the mechanical valve is a mechanical flapper or a mechanical baffle; and the solenoid valve is a miniature solenoid valve.

In addition, the present invention further provides a method for detecting nucleic acids, the method comprises the following steps:
(1) mixing a DNA to be tested with a PCR reaction solution in a microfluidic tank to obtain a mixed solution;
(2) subjecting the mixed solution obtained in step (1) to an isothermal amplification reaction in the microfluidic tank, and after the reaction is completed, adding a nuclease into the microfluidic tank, and cleaving a product obtained by the isothermal amplification reaction into a DNA fragment of a predetermined length;
(3) reacting the DNA fragment of a predetermined length obtained in step (2) with a capture chip containing a capture DNA to obtain a capture chip containing the DNA fragment of a predetermined length;
(4) after the reaction is completed, washing away an unbound DNA fragment with a cleaning solution;
(5) reacting the capture chip containing the DNA fragment of a predetermined length obtained in step (3) with a DNA-modified magnetic bead; and
(6) detecting a signal by a magneto-sensitive detector.

Further, the method for detecting nucleic acids comprises the following steps:
(1) placing the PCR reaction solution in the microfluidic tank, opening a valve on a fourth micro-channel which connects a reagent inlet of the microfluidic tank with a reagent outlet of a receiving chamber for DNA to be tested, so that the DNA to be tested flows into the microfluidic tank to obtain a mixed solution of the DNA to be tested and the PCR reaction solution;
(2) subjecting the mixed solution obtained in step (1) to an isothermal amplification reaction in the microfluidic tank, and after the reaction is completed, opening a valve on a fifth micro-channel which connects the reagent inlet of the microfluidic tank with a reagent outlet of a nuclease receiving chamber, adding a nuclease into the microfluidic tank, and cleaving a product obtained by the isothermal amplification reaction into a DNA fragment of a predetermined length;
(3) opening a valve on a first micro-channel which connects a reagent outlet of the microfluidic tank with an inlet of a capture chip storage chamber, so that the DNA fragment of a predetermined length obtained in step (2) flows into the capture chip storage chamber and reacts with the capture chip containing the capture DNA to obtain the capture chip containing the DNA fragment of a predetermined length;
(4) after the reaction is completed, opening a valve on a third micro-channel which connects a cleaning solution storage chamber with the inlet of the capture chip storage chamber, so that a cleaning solution flows into the capture chip storage chamber to wash away the unbound DNA fragment;
(5) opening a valve on a second micro-channel which connects a DNA-modified magnetic bead storage chamber with the inlet of the capture chip storage chamber, so that the DNA-modified magnetic bead flows into the capture chip storage chamber and reacts with the capture chip containing the DNA fragment of a predetermined length obtained in step (3); and
(6) detecting a signal by the magneto-sensitive detector.

Compared with the prior art, the beneficial effects of the present invention are: the device for detecting nucleic acids of the present invention is provided with a microfluidic tank, and an isothermal amplification reaction is carried out in the microfluidic tank, so that the reaction speed is fast, and the flow channel design is simplified with respect to PCR reactions; and the present invention further uses a magneto-sensitive detector, and thus the signal is stably output and does not attenuate over time.

### BRIEF DESCRIPTION OF THE DRAWINGS

FIG. 1 is a structural schematic diagram of a sample processor in a device for detecting nucleic acids according to an embodiment 1 of the present invention; and

FIG. 2 is a structural schematic diagram of a sample processor in a device for detecting nucleic acids according to an embodiment 2 of the present invention.

In the figures, 1 is a microfluidic tank, 2 is a temperature controller, 301 is a capture chip storage chamber, 302 is a DNA-modified magnetic bead storage chamber, 303 is a cleaning solution storage chamber, 4 is a receiving chamber for DNA to be tested, 5 is a nuclease receiving chamber, 601 is a first valve, 602 is a second valve, 603 is a third valve, 604 is a fourth valve, 605 is a fifth valve, 606 is a sixth valve, 701 is a first micro-channel, 702 is a second micro-channel, 703 is a third micro-channel, 704 is a fourth micro-channel, 705 is a fifth micro-channel, 8 is a pressurizer, 9 is a DNA extraction chamber, 10 is an RNA extraction chamber, 11 is a reverse transcription reagent storage chamber, and 12 is a waste solution cell.

### DETAILED DESCRIPTION OF EMBODIMENTS

For better illustration of the object, the technical solution and the advantages of the present invention, the present invention will be further illustrated below in conjunction with the accompanying drawings and specific embodiments.

### Embodiment 1

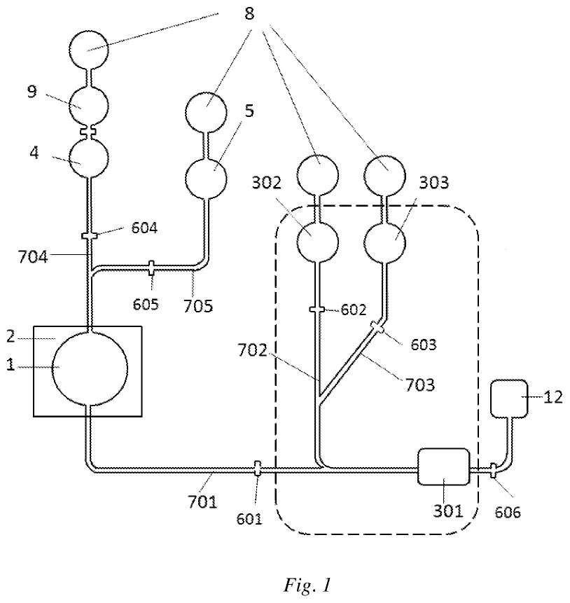

A device for detecting nucleic acids according to an embodiment of the present invention comprises a sample processor and a magneto-sensitive detector, wherein the sample processor has a structure as shown in FIG. 1, comprising a microfluidic tank 1, a temperature controller 2, a capture chip storage chamber 301, a DNA-modified magnetic bead storage chamber 302 and a cleaning solution storage chamber 303;
the microfluidic tank 1 is provided with a reagent inlet and a reagent outlet; the temperature controller 2 is disposed on the microfluidic tank 1;
an inlet of the capture chip storage chamber 301 is in communication with the reagent outlet of the microfluidic tank 1 through a first micro-channel 701, is in communication with the DNA-modified magnetic bead storage chamber 302 through a second micro-channel 702, and is in communication with the cleaning solution storage chamber 303 through a third micro-channel 703; and the first micro-channel 701 is provided with a first valve 601, the second micro-channel 702 is provided with a second valve 602, the third micro-channel 703 is provided with a third valve 603, and the first valve 601, the second valve 602 and the third valve 603 are used to control a reagent in the microfluidic tank 1, DNA-modified magnetic beads and a cleaning solution to flow into the capture chip storage chamber 301, respectively.

The magneto-sensitive detector comprises a magnetic sensor and a groove for accommodating the capture chip storage chamber 301, the capture chip storage chamber 301 is inserted inside the groove, and the magnetic sensor senses DNA-modified magnetic beads in the capture chip storage chamber 301 and converts magnetic signals of the DNA-modified magnetic beads into electrical signals.

The device for detecting nucleic acids of the present invention is provided with the microfluidic tank 1, and an isothermal amplification reaction is carried out in the microfluidic tank 1, so that the reaction speed is fast, and the flow channel design is simplified with respect to PCR reactions. Prior to use, the capture DNA is previously formed in the capture chip storage chamber 301. Then, the magneto-sensitive detector is disposed around the capture DNA, or around the substrate where the capture DNA is immobilized, or on the reverse side of the capture DNA via the substrate. When in use, the DNA to be tested and the PCR reaction solution can be first added into the microfluidic tank 1, and after the isothermal amplification reaction, the nuclease is added for cleaving so as to obtain a reaction product; and after the reaction product in the microfluidic tank 1 enters the capture chip storage chamber 301, the reaction product is captured by the capture DNA in the capture chip storage chamber 301, the unbound DNA is washed away with the cleaning solution in the cleaning solution storage chamber 303, the DNA to be tested further reacts with magnetic beads having a linker DNA in the

DNA-modified magnetic bead storage chamber 302, and detecting the DNA to be tested can be carried out by detecting a magnetic signal. Among them, said magnetic beads having a linker DNA refer to substances that bind to the DNA generated from the substrate where GMR is located and have magnetic beads attached to the end. By means of the magnetic signal detection, the signal is stably output and does not attenuate over time. The detector is a magneto-sensitive detector, such as a GMR (Giant Magneto Resistive) detector or TMR (Tunnel Magneto Resistive) detector.

In order to ensure the use of the device for detecting nucleic acids of the present invention, the following operations may be carried out in sequence: the reaction product in the microfluidic tank 1 first reacts with the capture DNA in the capture chip storage chamber 301, then the unbound DNA is washed away, and finally the reaction product in the microfluidic tank 1 reacts with the magnetic beads having a linker DNA. It is required that valves be provided to allow the reagent in the microfluidic tank 1, the DNA-modified magnetic beads and the cleaning solution to separately flow into the capture chip storage chamber 301.

Further, the sample processor for detecting nucleic acids of the present invention further comprises a receiving chamber for DNA to be tested 4 and a nuclease receiving chamber 5, and both of the receiving chamber for DNA to be tested 4 and the nuclease receiving chamber 5 is provided with a reagent inlet and a reagent outlet; the reagent inlet of the microfluidic tank 1 is in communication with the reagent outlet of the receiving chamber for DNA to be tested 4 through a fourth micro-channel 704 and is in communication with the reagent outlet of the nuclease receiving chamber 5 through a fifth micro-channel 705; and the fourth micro-channel 704 is provided with a fourth valve 604, the fifth micro-channel 705 is provided with a fifth valve 605, and the fourth valve 604 and the fifth valve 605 are used to control the DNA to be tested and the nuclease to flow to the microfluidic tank 1, respectively.

Further, both of the receiving chamber for DNA to be tested 4 and the nuclease receiving chamber 5 are disposed above the microfluidic tank 1; and the capture chip storage chamber 301 is located below the DNA-modified magnetic bead storage chamber 302 and the cleaning solution storage chamber 303, and the height of the capture chip storage chamber 301 is not higher than that of the microfluidic tank 1. The arrangement of the components according the specific orientation facilitates smooth flow of the reagent from top to bottom. Of course, it is also possible to allow the reagent to flow according to a predetermined flow path under an external force.

Further, the sample processor of the present embodiment further comprises a pressurizer 8, and the pressurizer 8 is respectively connected to the receiving chamber for DNA to be tested 4, the nuclease receiving chamber 5, the DNA-modified magnetic bead storage chamber 302 and the cleaning solution storage chamber 303. The pressurizer 8 can supply pressure to the receiving chamber for DNA to be tested 4, the nuclease receiving chamber 5, the DNA-modified magnetic bead storage chamber 302 and the cleaning solution storage chamber 303, that is, to provide a driving force for the reagent flow. The pressurizer 8 can be driven by the compressed air therein or by a mechanical pressure.

In order to directly carry out the DNA extraction process in the device of the present invention, the sample processor further comprises a DNA extraction chamber 9 having a DNA extraction solution therein, and the DNA extraction chamber 9 is in communication with the reagent inlet of the receiving chamber for DNA to be tested 4. In order to simultaneously supply a driving force to the DNA extraction chamber 9 and the receiving chamber for DNA to be tested 4 by the pressurizer 8, the DNA extraction chamber 9 is disposed between the receiving chamber for DNA to be tested 4 and the pressurizer 8. However, it should be noted that, instead of arranging the DNA extraction chamber 9 between the receiving chamber for DNA to be tested 4 and the pressurizer 8, the DNA extraction chamber and the pressurizer 8 may be respectively in communication with the reagent inlet of the receiving chamber for DNA to be tested 4.

Further, the temperature controller 2 comprises a heating body, a temperature sensor electrically connected to the heating body and detecting the temperature of the heating body, and a temperature control unit electrically connected to the heating body and controlling the temperature of the heating body; the temperature sensor is further electrically connected to the temperature control unit for transferring the detected temperature of the heating body to the temperature control unit; and the heating body is disposed on the microfluidic tank 1. The device of the present invention can use a temperature controller commonly used in the art, and the specific structure of the temperature controller is not shown in FIG. 1. The heating body may be strip-shaped or sheet-shaped.

In order to facilitate the collection of the cleaning solution or other waste solution, the sample processor further comprises a waste solution cell 12, the waste solution cell 12 is in communication with the outlet of the capture chip storage chamber 301, and the pipe through which the waste solution cell 12 is in communication with the outlet of the capture chip storage chamber 301 is provided with a sixth valve 606.

Further, in this embodiment, the valve is a mechanical valve or a solenoid valve. Preferably, the mechanical valve is a mechanical flapper or a mechanical baffle; and the solenoid valve is a miniature solenoid valve.

The method of using the device for detecting nucleic acids in this embodiment (i.e., the method for detecting nucleic acids of the present invention) comprises:
(1) extracting DNA in the DNA extraction chamber 9 to obtain DNA to be tested;
(2) allowing the DNA to be tested obtained in step (1) to enter the receiving chamber for DNA to be tested 4 from the DNA extraction chamber 9;
(3) placing a PCR reaction solution in the microfluidic tank 1, opening the fourth valve 604 on the fourth micro-channel 704 through which the reagent inlet of the microfluidic tank 1 is in communication with the reagent outlet of the receiving chamber for DNA to be tested 4, so that the DNA to be tested flows into the microfluidic tank 1 so as to obtain a mixed solution of the DNA to be tested and the PCR reaction solution;
(4) subjecting the mixed solution obtained in step (3) to an isothermal amplification reaction in the microfluidic tank, and after the reaction is completed, opening the fifth valve 605 on the fifth micro-channel 705 through which the reagent inlet of the microfluidic tank 1 is in communication with the reagent outlet of the nuclease receiving chamber 5, adding nuclease into the microfluidic tank 1, and cleaving the product obtained by the isothermal amplification reaction into DNA fragments of a predetermined length;
(5) opening the first valve 601 on the first micro-channel 701 through which the reagent outlet of the microfluidic tank 1 is in communication with the inlet of the capture chip storage chamber 301, so that the DNA fragments of a predetermined length obtained in step (4) flow into the capture chip storage chamber 301 and react with capture chips containing capture DNA so as to obtain capture chips containing DNA fragments of a predetermined length;
(6) after the reaction is completed, opening the third valve 603 on the third micro-channel 703 through which the cleaning solution storage chamber 303 is in communication with the inlet of the capture chip storage chamber 301, so that the cleaning solution flows into the capture chip storage chamber 301 to wash away unbound DNA fragments;
(7) opening the second valve 602 on the second micro-channel 702 through which the DNA-modified magnetic bead storage chamber 302 is in communication with the inlet of the capture chip storage chamber 301, so that the DNA-modified magnetic beads flow into the capture chip storage chamber and react with the capture chips containing DNA fragments of a predetermined length obtained in step (5); and
(8) detecting signals by the magneto-sensitive detector.

### Embodiment 2

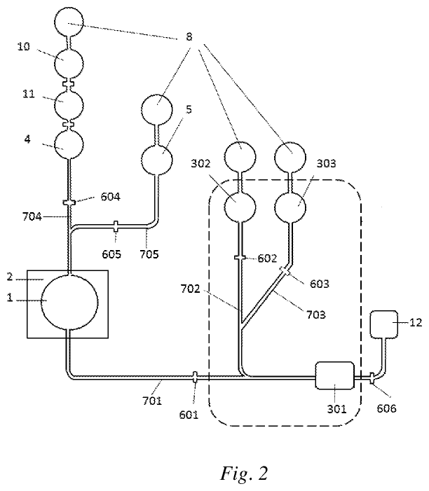

A device for detecting nucleic acids of an embodiment of the present invention differs from embodiment 1 merely in that the sample processor is different. The sample processor of the device for detecting nucleic acids in this embodiment is as shown in FIG. 2, which differs from the sample processor of embodiment 1 in that the sample processor of the device for detecting nucleic acids of the present embodiment does not comprise a DNA extraction chamber 9 having a DNA extraction solution therein, but the sample processor of the device for detecting nucleic acids of the present embodiment further comprises an RNA extraction chamber 10 having a RNA extraction solution therein and a reverse transcription reagent storage chamber 11, and the reverse transcription reagent storage chamber 11 is respectively in communication with the reagent outlet of the RNA extraction chamber 10 and the reagent inlet of the receiving chamber for DNA to be tested 4; and in the present embodiment, the pressurizer 8 is connected to the RNA extraction chamber 10.

The device for detecting nucleic acids of the present embodiment can extract RNA and carry out RNA assay. The method for using the device for detecting nucleic acids of the present embodiment differs from embodiment 1 merely in that step (1) is different. When the device for detecting nucleic acids of the present embodiment is used, step (1) comprises extracting RNA in the RNA extraction chamber 10, and allowing the extracted RNA to enter the reverse transcription reagent storage chamber 11 from the RNA extraction chamber 10 and carry out reverse transcription so as to obtain DNA to be tested.

Finally, it should be stated that the above embodiments are merely used for illustrating the technical solution of the present invention rather than limiting the scope of protection of the present invention; and although the present invention has been illustrated in detail with reference to the preferred embodiments, a person skilled in the art should understand that modifications or equivalent substitutions may be made to the technical solution of the present invention without departing from the spirit and scope of the technical solution of the present invention.

1. A device for detecting nucleic acids, wherein the device comprises a sample processor and a magneto-sensitive detector, the sample processor comprises a microfluidic tank, a temperature controller, a capture chip storage chamber, a DNA-modified magnetic bead storage chamber and a cleaning solution storage chamber;
the microfluidic tank is provided with a reagent inlet and a reagent outlet; the temperature controller is disposed on the microfluidic tank;
an inlet of the capture chip storage chamber is in communication with the reagent outlet of the microfluidic tank through a first micro-channel, is in communication with the DNA-modified magnetic bead storage chamber through a second micro-channel, and is in communication with the cleaning solution storage chamber through a third micro-channel; the first micro-channel, the second micro-channel and the third micro-channel are provided with valves to control a reagent in the microfluidic tank, a DNA-modified magnetic bead and a cleaning solution to flow into the capture chip storage chamber, respectively; and
the magneto-sensitive detector comprises a magnetic sensor and a groove for accommodating the capture chip storage chamber, the capture chip storage chamber is inserted inside the groove, and the magnetic sensor senses the DNA-modified magnetic bead in the capture chip storage chamber and converts a magnetic signal of the DNA-modified magnetic bead into an electrical signal.

2. A device for detecting nucleic acids, wherein the device comprises a sample processor and a magneto-sensitive detector, the sample processor comprises a microfluidic tank, a temperature controller, a capture chip storage chamber, a DNA-modified magnetic bead storage chamber and a cleaning solution storage chamber;
the microfluidic tank is provided with a reagent inlet and a reagent outlet; the temperature controller is disposed on the microfluidic tank;
an inlet of the capture chip storage chamber is in communication with the reagent outlet of the microfluidic tank through a first micro-channel, is in communication with the DNA-modified magnetic bead storage chamber through a second micro-channel, and is in communication with the cleaning solution storage chamber through a third micro-channel; the first micro-channel, the second micro-channel and the third micro-channel are provided with valves to control a reagent in the microfluidic tank, a DNA-modified magnetic bead and a cleaning solution to flow into the capture chip storage chamber, respectively; and
the magneto-sensitive detector comprises a magnetic sensor, and the magnetic sensor senses the DNA-modified magnetic bead in the capture chip storage chamber and converts a magnetic signal of the DNA-modified magnetic bead into an electrical signal.

3. The device for detecting nucleic acids as claimed in claim 1, wherein the sample processor further comprises a receiving chamber for DNA to be tested and a nuclease receiving chamber, and both of the receiving chamber for DNA to be tested and the nuclease receiving chamber are provided with a reagent inlet and a reagent outlet; the reagent inlet of the microfluidic tank is in communication with the reagent outlet of the receiving chamber for DNA to be tested through a fourth micro-channel and is in communication with the reagent outlet of the nuclease receiving chamber through a fifth micro-channel; and the fourth micro-channel and the fifth micro-channel are provided with valves to control a DNA to be tested and a nuclease to flow to the microfluidic tank, respectively.

4. The device for detecting nucleic acids as claimed in claim 3, wherein both of the receiving chamber for DNA to be tested and the nuclease receiving chamber are disposed above the microfluidic tank; and the capture chip storage chamber is located below the DNA-modified magnetic bead storage chamber and the cleaning solution storage chamber, and the height of the capture chip storage chamber is not higher than that of the microfluidic tank.

5. The device for detecting nucleic acids as claimed in claim 3, wherein the sample processor further comprises a pressurizer which is respectively connected to the receiving chamber for DNA to be tested, the nuclease receiving chamber, the DNA-modified magnetic bead storage chamber and the cleaning solution storage chamber.

6. The device for detecting nucleic acids as claimed in claim 3, wherein the sample processor further comprises a DNA extraction chamber having a DNA extraction solution therein, and the DNA extraction chamber is in communication with the reagent inlet of the receiving chamber for DNA to be tested.

7. The device for detecting nucleic acids as claimed in claim 3, wherein the sample processor further comprises an RNA extraction chamber having an RNA extraction solution therein and a reverse transcription reagent storage chamber, and the reverse transcription reagent storage chamber is respectively in communication with a reagent outlet of the RNA extraction chamber and the reagent inlet of the receiving chamber for DNA to be tested.

8. The device for detecting nucleic acids as claimed in claim 1, wherein the temperature controller comprises a heating body, a temperature sensor electrically connected to the heating body and detecting the temperature of the heating body, and a temperature control unit electrically connected to the heating body and controlling the temperature of the heating body; the temperature sensor is further electrically connected to the temperature control unit for transferring the detected temperature of the heating body to the temperature control unit; and the heating body is disposed on the microfluidic tank.

9. The device for detecting nucleic acids as claimed in claim 1, wherein the sample processor further comprises a waste solution cell that is in communication with an outlet of the capture chip storage chamber, and a pipe connecting the waste solution cell with the outlet of the capture chip storage chamber is provided with a valve.

10. The device for detecting nucleic acids as claimed in claim 1, wherein the valve is a mechanical valve or a solenoid valve.

11. The device for detecting nucleic acids as claimed in claim 10, wherein the mechanical valve is a mechanical flapper or a mechanical baffle; and the solenoid valve is a miniature solenoid valve.

12. The device for detecting nucleic acids as claimed in claim 2, wherein the magneto-sensitive detector is located within a groove for accommodating the capture chip storage chamber.

13. A method for detecting nucleic acids, wherein the method comprises the following steps:
(1) mixing a DNA to be tested with a PCR reaction solution in a microfluidic tank to obtain a mixed solution;
(2) subjecting the mixed solution obtained in step (1) to an isothermal amplification reaction in the microfluidic tank, and after the reaction is completed, adding a nuclease into the microfluidic tank, and cleaving a product obtained by the isothermal amplification reaction into a DNA fragment of a predetermined length;
(3) reacting the DNA fragment of a predetermined length obtained in step (2) with a capture chip containing a capture DNA to obtain a capture chip containing the DNA fragment of a predetermined length;
(4) after the reaction is completed, washing away an unbound DNA fragment with a cleaning solution;
(5) reacting the capture chip containing the DNA fragment of a predetermined length obtained in step (3) with a DNA-modified magnetic bead; and
(6) detecting a signal by a magneto-sensitive detector.

14. The method for detecting nucleic acids as claimed in claim 13, wherein the method comprises the following steps:
(1) placing the PCR reaction solution in the microfluidic tank, opening a valve on a fourth micro-channel which connects a reagent inlet of the microfluidic tank with a reagent outlet of a receiving chamber for DNA to be tested, so that the DNA to be tested flows into the microfluidic tank to obtain a mixed solution of the DNA to be tested and the PCR reaction solution;
(2) subjecting the mixed solution obtained in step (1) to an isothermal amplification reaction in the microfluidic tank, and after the reaction is completed, opening a valve on a fifth micro-channel which connects the reagent inlet of the microfluidic tank with a reagent outlet of a nuclease receiving chamber, adding a nuclease into the microfluidic tank, and cleaving a product obtained by the isothermal amplification reaction into a DNA fragment of a predetermined length;
(3) opening a valve on a first micro-channel which connects a reagent outlet of the microfluidic tank with an inlet of a capture chip storage chamber, so that the DNA fragment of a predetermined length obtained in step (2) flows into the capture chip storage chamber and reacts with the capture chip containing the capture DNA to obtain the capture chip containing the DNA fragment of a predetermined length;
(4) after the reaction is completed, opening a valve on a third micro-channel which connects a cleaning solution storage chamber with the inlet of the capture chip storage chamber, so that a cleaning solution flows into the capture chip storage chamber to wash away the unbound DNA fragment;
(5) opening a valve on a second micro-channel which connects a DNA-modified magnetic bead storage chamber with the inlet of the capture chip storage chamber, so that the DNA-modified magnetic bead flows into the capture chip storage chamber and reacts with the capture chip containing the DNA fragment of a predetermined length obtained in step (3); and
(6) detecting a signal by the magneto-sensitive detector.

15. The device for detecting nucleic acids as claimed in claim 2, wherein the sample processor further comprises a receiving chamber for DNA to be tested and a nuclease receiving chamber, and both of the receiving chamber for DNA to be tested and the nuclease receiving chamber are provided with a reagent inlet and a reagent outlet; the reagent inlet of the microfluidic tank is in communication with the reagent outlet of the receiving chamber for DNA to be tested through a fourth micro-channel and is in communication with the reagent outlet of the nuclease receiving chamber through a fifth micro-channel; and the fourth micro-channel and the fifth micro-channel are provided with valves to control a DNA to be tested and a nuclease to flow to the microfluidic tank, respectively.

16. The device for detecting nucleic acids as claimed in claim 2, wherein both of the receiving chamber for DNA to be tested and the nuclease receiving chamber are disposed above the microfluidic tank; and the capture chip storage chamber is located below the DNA-modified magnetic bead storage chamber and the cleaning solution storage chamber, and the height of the capture chip storage chamber is not higher than that of the microfluidic tank; or
wherein the sample processor further comprises a pressurizer which is respectively connected to the receiving chamber for DNA to be tested, the nuclease receiving chamber, the DNA-modified magnetic bead storage chamber and the cleaning solution storage chamber.

17. The device for detecting nucleic acids as claimed in claim 2, wherein the sample processor further comprises a DNA extraction chamber having a DNA extraction solution therein, and the DNA extraction chamber is in communication with the reagent inlet of the receiving chamber for DNA to be tested; or
wherein the sample processor further comprises an RNA extraction chamber having an RNA extraction solution therein and a reverse transcription reagent storage chamber, and the reverse transcription reagent storage chamber is respectively in communication with a reagent outlet of the RNA extraction chamber and the reagent inlet of the receiving chamber for DNA to be tested.

18. The device for detecting nucleic acids as claimed in claim 2, wherein the temperature controller comprises a heating body, a temperature sensor electrically connected to the heating body and detecting the temperature of the heating body, and a temperature control unit electrically connected to the heating body and controlling the temperature of the heating body; the temperature sensor is further electrically connected to the temperature control unit for transferring the detected temperature of the heating body to the temperature control unit; and the heating body is disposed on the microfluidic tank.

19. The device for detecting nucleic acids as claimed in claim 2, wherein the sample processor further comprises a waste solution cell that is in communication with an outlet of the capture chip storage chamber, and a pipe connecting the waste solution cell with the outlet of the capture chip storage chamber is provided with a valve.

20. The device for detecting nucleic acids as claimed in claim 2, wherein the valve is a mechanical valve or a solenoid valve; and
wherein the mechanical valve is a mechanical flapper or a mechanical baffle; and the solenoid valve is a miniature solenoid valve.
